(19) United States
(12) Patent Application Publication
(10) Pub. No.: US 2008/0208110 A1
(43) Pub. Date: Aug. 28, 2008

(54) Infusion system with injection site

(76) Inventor: Robert J. Sanchez, Oceanside, CA (US)

Correspondence Address: ALCON, IP LEGAL, TB4-8, 6201 SOUTH FREEWAY, FORT WORTH, TX, 76134, US

(21) Appl. No.: 11/710,693

(22) Filed: Feb. 26, 2007

(51) Int. Cl. A61M 5/14

(52) U.S. Cl. 604/26; 604/31; 604/521

(57) ABSTRACT

An infusion system with a needleless or needle-requiring injection site placed downstream of a fluid selection stopcock valve and upstream of the infusion cannula. In an alternate embodiment the fluid selection stopcock valve and injection site may be formed as a single consumable unit.

### FIELD

The present invention pertains to a system used during ophthalmic surgery to facilitate the exchange of fluids within the interior of the eye; more particularly, the present invention pertains to a system for the infusion of fluid into the posterior segment of the eye.

### BACKGROUND

During ophthalmic surgery, and in particular vitreoretinal surgery, the need arises to exchange the fluid within the eye with another fluid, typically a gas. A system by which such exchange may be accomplished is shown in FIG. 1. Therein it may be seen that an air infusion line and a liquid infusion line are connected to an automatic stopcock valve as described in co-pending U.S. patent application Ser. No. 11/522,648 filed Sep. 18, 2006 by the assignee of the instant application and incorporated herein by reference.

The exchange of fluid from within the interior of the eye with a gas, for example air, is typically performed by use of the automatic stopcock valve. Specifically, the automatic stopcock valve is opened by air pressure. This opening of the automatic stopcock valve to the flow of pressurized air stops the flow of the intraocular irrigating solution (e.g. BSS PLUS® intraocular irrigating solution available from Alcon Laboratories, Inc. of Fort Worth, Tex.) through the liquid infusion line and starts the flow of air through the air infusion line. For the eye surgeon—when time comes to stop the flow of surgical infusion solution and begin the flow of air—the eye surgeon will make the exchange of fluids by activating the flow of pressurized air using either a computer or manual control.

Downstream from the automatic stopcock valve is a connection for an infusion cannula. Downstream from the connection for the infusion cannula is the infusion cannula itself which conducts fluid into the eye of the patient.

During vitreoretinal surgery, the need often arises to inject a special fluid or medication into the eye. One of three techniques is typically employed to inject the special fluid or medication through the infusion cannula into the eye, as follows:

1. The special fluid or medication is injected using a manual, three way stopcock valve as shown in FIG. 1A. The system shown in FIG. 1A is a modification of the system shown in FIG. 1. Use of the three way stopcock valve requires the eye surgeon to manually actuate the three way stopcock valve before the special fluid or medication is injected.

2. The infusion line is disconnected at the connection point for the infusion cannula tubing set and the special fluid or medication is injected at the now dis-assembled connection point. The use of this technique requires that the liquid infusion line be clamped to prevent leakage of infusion fluid.

3. A small gauge needle is used by the surgeon to directly inject the special fluid or medication into the posterior segment of the eye. However, intraocular injections sometimes result in complications for the patient.

Whichever of the foregoing techniques is used, the eye surgeon is presented with an added degree of difficulty and a time delay when the need arises to inject a special fluid into the eye of the patient during ophthalmic surgery.

Accordingly, a need remains in the art for a system which will reduce the difficulty and the time required when the need arises to inject a special fluid or medication into the eye of a patient during ophthalmic surgery.

### SUMMARY

According to the present invention the difficulty and time required to inject a special fluid or medication into the eye of a patient during ophthalmic surgery is reduced.

The present invention is an infusion system having an injection site for the infusion of fluids during ophthalmic surgery. The injection site may be placed downstream from a manual stopcock valve, downstream from an automatic stopcock valve, or incorporated into a consumable automatic stopcock valve unit.

### BRIEF DESCRIPTION OF DRAWING FIGURES

A better description of the infusion system with injection site of the present invention may be had by reference to attached drawing figures wherein:

FIG. 1 is schematic of a prior art infusion system using an automatic stopcock valve;

FIG. 1A is a schematic of a prior art infusion system similar to the prior art infusion system shown in FIG. 1 but further including a manual, three way stopcock valve;

FIG. 2 is a schematic of an infusion system including an injection site downstream from a stopcock valve;

FIG. 3 is a schematic of an infusion system including an injection site downstream from a an automatic stopcock valve;

FIG. 4 is a schematic of an infusion system including an injection site incorporated into a consumable unit with an automatic stopcock valve.

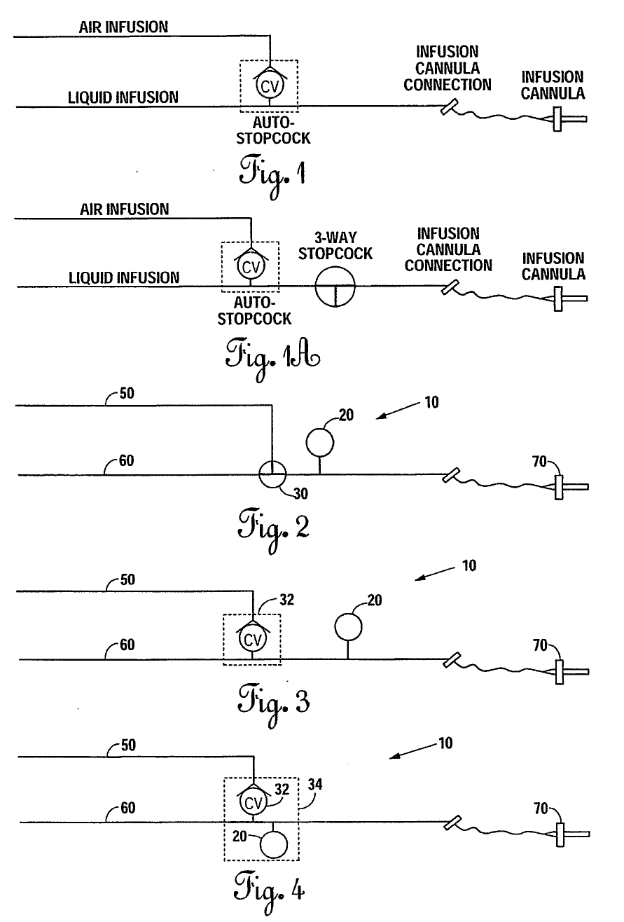

### DESCRIPTION OF THE EMBODIMENTS

Needleless injection sites, or needle-requiring injection sites, are products well know to the medical device industry. Such injection sites may be embodied in a Y fitting or T fitting which may be inserted into the type of plastic tubing typically used in a fluid infusion system, or which may include a luer lock at its ends for connection to the sections of the plastic tubing. A needleless injection site typically requires the use of a male luer tipped syringe. A needle-requiring injection site typically requires the use of a needle-requiring syringe. Still other injection sites are available and are well know to those of ordinary skill in the art.

According to the embodiment shown in FIG. 2, an injection site 20 is added to an infusion system 10 downstream from a manual stopcock valve 30. Injection site 20 may be a needleless injection site or a needle-requiring injection site. Stopcock valve 30 permits the selective infusion of either gas or liquid through a gas infusion line 50 or a liquid infusion line 60 respectively. By use of injection site 20 placed downstream from stopcock 30 and upstream from infusion cannula 70, the eye surgeon has the option of adding a special fluid or medication into infusion system 10 through injection site 20 with or without stopping the flow of fluid through liquid infusion line 60 to infusion cannula 70 and without having to disassemble infusion system 10.

In the embodiment shown in FIG. 3, injection site 20 is placed downstream from an automatic stopcock valve 32, which as previously indicated is described in co-pending U.S. patent application Ser. No. 11/522,648. By use of injection site 20 placed downstream from automatic stopcock valve 32 and upstream from infusion cannula 70, the eye surgeon may add a special fluid or medication to infusion system 10 with or without stopping the flow of fluid through liquid infusion line 60 to infusion cannula 70, without having to disassemble infusion system 10, and without having to manually change the condition of any valve.

In the embodiment shown in FIG. 4, injection site 20 is incorporated into a consumable unit 34. Consumable unit 34 includes automatic stopcock valve 32. As in the embodiment of FIG. 3, the eye surgeon is able to add special fluid or a medication to the infusion system 10 with or without stopping the flow of fluid through liquid infusion line 60 to infusion cannula 70, without having to disassemble infusion system 10, and without having to manually change the condition of any valve.

Thus, according to the present invention the difficulty and time associated with the infusion of a special fluid or medication into the eye of a patient during ophthalmic surgery is reduced.

The present invention is illustrated herein by example, and various modifications may be made by a person of ordinary skill in the art. For example, gas infusion line 50 and liquid infusion line 60 may be fluid infusion lines that each conduct a different gas, or each conduct a different liquid. As another example, infusion line 50 may conduct a liquid, and infusion line 60 may conduct a gas.

While the present invention has been described according to its preferred and alternate embodiments, those of ordinary skill in the art will understand that other embodiments have been enabled by the foregoing disclosure. Such other embodiments shall be included within the scope and meaning of the appended claims.

What is claimed is:

1. A system for the infusion of fluids into the eye of a patient during eye surgery, said system comprising:
a first fluid infusion line;
a second fluid infusion line;
said first and second fluid infusion lines being connected to a stopcock valve for selective infusion of either said first fluid or said second fluid;
an infusion cannula being connected to said stopcock valve;
an injection site placed downstream from said stopcock valve and upstream from said infusion cannula.

2. The system as defined in claim 1 wherein said injection site is a needleless injection site.

3. The system as defined in claim 1 wherein said injection site is a needle-requiring injection site.

4. The system as defined in claim 1 wherein said stopcock valve is a manual stopcock valve.

5. The system as defined in claim 1 wherein said stopcock valve is an automatic stopcock valve.

6. The system as defined in claim 5 wherein said automatic stopcock valve and said injection site are formed as a consumable unit.

7. The system as defined in claim 1 wherein said first fluid infusion line is a liquid infusion line and said second fluid infusion line is a gas infusion line.

8. The system as defined in claim 1 wherein said first fluid infusion line and said second fluid infusion line are liquid infusion lines.

9. The system as defined in claim 1 wherein said first fluid infusion line and said second fluid infusion line are gas infusion lines.

10. A method of providing for the injection of a special fluid or medication through an infusion system into the eye of a patient during eye surgery, said method comprising the steps of:
conducting a first fluid through a first infusion line to a stopcock valve;
conducting a second fluid through a second infusion line to said stopcock valve;
selecting between said first fluid and said second fluid using said stopcock valve;
conducting either said first fluid or said second fluid to the eye of the patient through an infusion cannula;
placing an injection site downstream from said stopcock valve and upstream from said infusion cannula.

11. The method as defined in claim 10 wherein said injection site is a needleless injection site.

12. The method as defined in claim 10 wherein said injection site is a needle-requiring injection site.

13. The method as defined in claim 10 wherein said stopcock valve is a manual stopcock valve.

14. The method as defined in claim 10 wherein said stopcock valve is an automatic stopcock valve.

15. The method as defined in claim 14 wherein said automatic stopcock valve and said injection site are formed as a consumable unit.

16. The method as defined in claim 10 wherein said first fluid is a liquid and said second fluid is a gas.

17. The method as defined in claim 10 wherein said first fluid and said second fluid are liquids.

18. The method as defined in claim 10 wherein said first fluid and said second fluid are gases.
